(19) United States
(12) Patent Application Publication
(10) Pub. No.: US 2013/0211451 A1
(43) Pub. Date: Aug. 15, 2013

(54) BONE ANCHOR AND RELATED INSTRUMENTATION AND METHODS

(71) Applicant: Anulex Technologies, Inc., (US)

(72) Inventors: Lawrence W. Wales, Maplewood, MN (US); Ishmael Bentley, Eagan, MN (US); Dale Brady, New Brighton, MN (US)

(73) Assignee: Anulex Technologies, Inc., Minnetonka, MN (US)

(21) Appl. No.: 13/673,626

(22) Filed: Nov. 9, 2012

Related U.S. Application Data
(60) Provisional application No. 61/596,804, filed on Feb. 9, 2012.

(51) Int. Cl. A61B 17/04

(52) U.S. Cl. CPC A61B 17/0401
USPC 606/232

(57) ABSTRACT

An implantable bone anchor assembly comprises an outer anchor tube and a tubular insert. The outer anchor tube defines a longitudinal axis of the bone anchor assembly and a longitudinal channel. The outer anchor tube has a first portion including a first end, and a plurality of fingers circumferentially spaced from one another about the longitudinal axis. Each of the fingers extends from the first portion and has a free end, wherein the fingers are configured to project laterally at an oblique angle relative to the longitudinal axis, and are further configured to be deflectable radially inward to allow the anchor tube to be disposed within a tubular cannula for deployment. The tubular insert has a head having an opening therein, a shank extending from the head having an open end, and a longitudinal bore extending from the opening in the head to the open end of the shank. The longitudinal bore is configured to receive a portion of a suture assembly for an orthopedic treatment. The shank is sized to be received in the longitudinal channel, and the outer anchor tube and the tubular insert are configured to be locked together upon complete insertion of the shank into the longitudinal channel.

### CROSS-REFERENCE TO RELATED APPLICATION

This application claims the benefit of U.S. Provisional Application No. 61/596,804, filed Feb. 9, 2012, which is herein incorporated by reference in its entirety.

### TECHNICAL FIELD

The present invention relates to implants and instrumentation for surgical procedures. More specifically, the invention relates to a bone anchor and related suture assemblies and method of using the foregoing.

### BACKGROUND

Various conventional bone anchors are known for use in orthopedic repair procedures. There is a continuing need for improved bone anchors and related instrumentation.

### SUMMARY

In Example 1, an embodiment of the present invention is an implantable bone anchor assembly comprising an outer anchor tube and a tubular insert. The outer anchor tube defines a longitudinal axis of the bone anchor assembly and a longitudinal channel. The outer anchor tube has a first portion including a first end, and a plurality of fingers circumferentially spaced from one another about the longitudinal axis, each of the fingers extending from the first portion and having a free end. The fingers are configured to project laterally at an oblique angle relative to the longitudinal axis, and are further configured to be deflectable radially inward to allow the anchor tube to be disposed within a tubular cannula for deployment. The tubular insert has a head including an opening therein, a shank extending from the head having an open end, and a longitudinal bore extending from the opening in the head to the open end of the shank, the shank sized to be received in the longitudinal channel. The outer anchor tube and the tubular insert are configured to be secured together upon complete insertion of the shank into the longitudinal channel.

In Example 2, the bone anchor assembly of Example 1, wherein the opening in the head and the open end of the shank are defined by radiused or chamfered edges.

In Example 3, the bone anchor assembly of either of Examples 1 or 2, wherein the head of the insert has a radial shoulder that abuts the first end of the outer anchor tube when the shank is fully inserted into the longitudinal channel of the outer anchor tube.

In Example 4, the bone anchor assembly of any of Examples 1-3, wherein the anchor tube includes a plurality of tabs configured to engage the insert to couple the insert and the outer anchor tube together when the shank is fully inserted into the longitudinal channel.

In Example 5, the bone anchor assembly of any of Examples 1-4, wherein the outer anchor tube is made from a shape memory alloy.

In Example 6, the bone anchor assembly of any of Examples 1-5, wherein the fingers of the outer anchor tube are configured to engage bone when implanted.

In Example 7, the bone anchor assembly of any of Examples 1-6, further comprising an adjustable suture assembly including a first portion extending within the bore of the insert and configured to engage the insert to resist withdrawal of the first portion from the bore of the insert after implantation.

In Example 8, the bone anchor assembly of any of Examples 1-7, wherein the outer anchor tube is a first outer anchor tube, and further comprising a second outer anchor tube having a longitudinal channel, and a plurality of fingers circumferentially spaced from one another about the longitudinal axis, each of the fingers of the second outer anchor tube having a free end and being configured to project laterally at an oblique angle relative to the longitudinal axis of the second outer anchor tube and to be deflectable radially inward to allow the second anchor tube to be disposed within a tubular cannula for deployment, wherein the shank of the insert is sized to be received within the longitudinal channel of the second outer anchor tube.

In Example 9, the bone anchor assembly of Example 8, wherein the shank includes a plurality of serrations along a portion of its length, each serration including a first surface extending at an oblique angle with respect to the longitudinal axis of the bone anchor assembly and a second surface extending generally orthogonal to the longitudinal axis and oriented generally toward the head of the insert, wherein the second outer anchor tube is disposed along a portion of the shank including the serrations, and wherein the serrations and the second outer anchor tube cooperate to permit relative translation of the insert and the second outer anchor tube in a first direction, and to inhibit relative translation of the insert and the second outer anchor tube in a second direction opposite the first direction.

In Example 10, the present invention is a fixation element for an orthopedic procedure, the fixation element comprising a bone anchor assembly and a suture assembly coupled to the bone anchor assembly. The bone anchor assembly includes an outer anchor tube and a tubular insert. The outer anchor tube defines a longitudinal axis of the bone anchor assembly and a longitudinal channel, and has a first portion including a first end, and a plurality of fingers circumferentially spaced from one another about the longitudinal axis. Each of the fingers extends from the first portion and has a free end, wherein the fingers are configured to project laterally at an oblique angle relative to the longitudinal axis, and are further configured to be deflectable radially inward to allow the anchor tube to be disposed within a tubular cannula for deployment. The tubular insert has a head including an opening therein, a shank extending from the head having an open end, and a longitudinal bore extending from the opening in the head to the open end of the shank, the shank sized to be received in the longitudinal channel. The outer anchor tube and the tubular insert are configured to be secured together upon complete insertion of the shank into the longitudinal channel. The suture assembly includes an adjustable suture loop and a knot configured for facilitating tightening of the suture loop. The bone anchor assembly is coupled to the adjustable suture loop.

In Example 11, the fixation element of Example 10, wherein the bone anchor assembly is slidably coupled to the suture loop.

In Example 12, the fixation element of either of Examples 10 or 11, further comprising a suture element connected to the bone anchor assembly and extending at least partially within the longitudinal bore of the insert, wherein the suture element is formed as a loop and is configured to slidably couple the bone anchor assembly to the adjustable suture loop.

In Example 13, the fixation element of any of Examples 10-12, further comprising a second bone anchor assembly including an outer anchor tube and a tubular insert, the outer anchor tube of the second bone anchor assembly defining a longitudinal axis of the second bone anchor assembly and a longitudinal channel, and having a first portion including a first end, and a plurality of fingers circumferentially spaced from one another about the longitudinal axis, each of the fingers extending from the first portion and having a free end, wherein the fingers are configured to project laterally at an oblique angle relative to the longitudinal axis, and are further configured to be deflectable radially inward to allow the anchor tube to be disposed within a tubular cannula for deployment. The tubular insert of the second bone anchor assembly has a head including an opening therein, a shank extending from the head having an open end, and a longitudinal bore extending from the opening in the head to the open end of the shank, the shank sized to be received in the longitudinal channel. The outer anchor tube and the tubular insert of the second bone anchor assembly are configured to be secured together upon complete insertion of the shank into the longitudinal channel. The second bone anchor assembly is coupled to the adjustable suture loop.

In Example 14, the fixation element of Example 13, wherein the bone anchor assembly and the second bone anchor assembly are slidably coupled to the adjustable suture loop.

In Example 15, the fixation element of either of Examples 13 or 14, wherein one of the bone anchor assembly and the second bone anchor assembly is slidably coupled to the adjustable suture loop, and wherein the other of the bone anchor assembly and the second bone anchor assembly is fixedly coupled to the adjustable suture loop.

In Example 16, the fixation element of Example 10, further comprising a tissue anchor coupled to the adjustable suture loop.

In Example 17, the fixation element of Example 16, wherein the tissue anchor is formed from a length of suture material, and wherein the adjustable suture loop passes through the length of suture material of the tissue anchor at a plurality of locations therein.

In Example 18, a method of deploying a fixation element for use in an orthopedic repair procedure. The method comprises forming a first bore within a first bone proximate tissue to be repaired, and positioning a cannula within the bore, the cannula releasably receiving at least a portion of a fixation element. The fixation element includes an outer anchor tube and a tubular insert. The outer anchor tube defines a longitudinal axis of the bone anchor assembly and a longitudinal channel, the outer anchor tube having a first portion including a first end, and a plurality of fingers circumferentially spaced from one another about the longitudinal axis, each of the fingers extending from the first portion and having a free end, wherein the fingers are configured to project laterally at an oblique angle relative to the longitudinal axis, and are further configured to be deflectable radially inward, wherein the cannula retains the fingers in the inward deflected configuration for delivery. The tubular insert has a head including an opening therein, a shank extending from the head having an open end, and a longitudinal bore extending from the opening in the head to the open end of the shank, the longitudinal bore configured to receive a portion of a suture assembly for an orthopedic treatment. The shank is sized to be received in the longitudinal channel, wherein the outer anchor tube and the tubular insert are configured to be locked together upon complete insertion of the shank into the longitudinal channel. The method further comprises removing the cannula while leaving the bone anchor assembly within the bore, wherein removing the cannula allows the fingers to project radially outward at an oblique angle to engage a surface within the bore.

In Example 19, the method of Example 18, wherein the fixation element further includes an adjustable suture assembly including a first portion extending at least partially within the longitudinal bore of the insert coupled to the insert, and a second portion extending from or coupled to the first portion and including an adjustable suture loop.

In Example 20, the method of either of Examples 18 or 19, wherein the fixation element further includes a second bone anchor assembly, and wherein the bone anchor assembly and the second bone anchor assembly are pre-loaded into the cannula prior to deployment of the bone anchor assembly and the second bone anchor assembly.

In Example 21, the method of claim 20, further comprising forming a second bore within the first bone or a second bone proximate tissue to be repaired, positioning the cannula into the second bore through the cannula, removing the cannula while leaving the second bone anchor assembly in the second bore.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is an elevation view of a bone anchor assembly according to one embodiment of the present invention.

FIG. 2 is a cross-sectional elevation view of the bone anchor assembly of FIG. 1 according to one embodiment of the present invention.

FIGS. 3-4 are elevation views showing the bone anchor assembly of FIG. 1 during implantation, according to one embodiment of the present invention.

FIGS. 5-10A/B are schematic illustrations of various bone anchor/suture assembly combinations according to various exemplary embodiments of the present invention.

FIGS. 11-13 are schematic illustrations of various bone anchor assemblies according to additional exemplary embodiments of the invention.

FIG. 14 is a schematic illustration of the skeletal system of a human hand showing exemplary therapeutic applications of various embodiments of the present invention.

FIG. 15 is a schematic illustration of the skeletal system of a human foot illustrating exemplary therapeutic applications of various embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

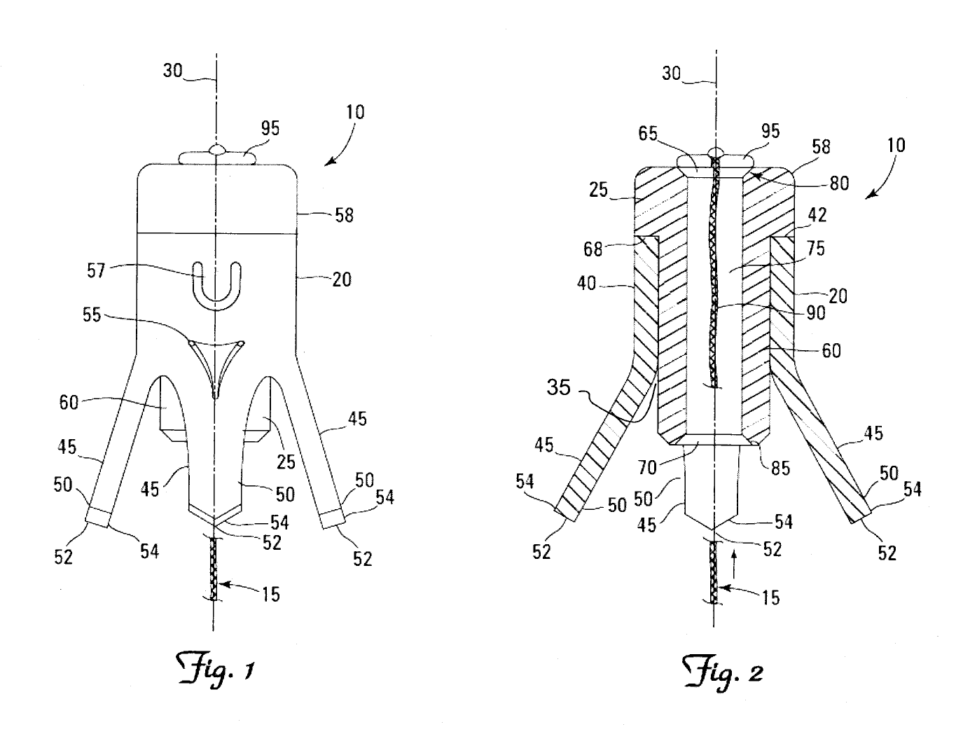

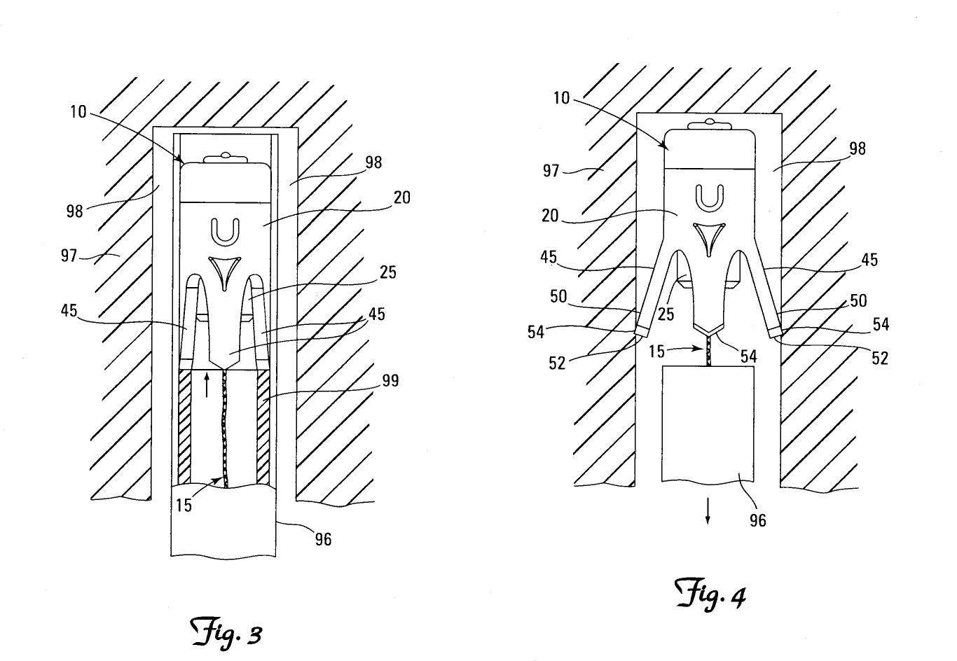

FIGS. 1 and 2 are outer and cross-sectional elevation views of a bone anchor assembly 10 and a suture element 15 according to one embodiment of the present invention. The bone anchor assembly 10 and suture element 15 are, in the various embodiments, particularly suited for orthopedic repair procedures such as, for example, crossover toe, hallux valgus, hammertoe, carpometacarpal arthroplasty, Bankart repair, intervertebral disc repair procedures, or other general orthopedic procedures. As such, the suture element 15 is connected to the bone anchor assembly 10, which when implanted in bone proximate the desired treatment site provides a firm anchoring point to resist tension applied to the suture element 15 both acutely during implantation and chronically after the procedure is complete. In various embodiments, the suture element 15 may include a pre-formed, adjustable suture loop (not shown) that can be interconnected with other suture elements or bone or soft tissue anchors depending on the desired repair technique.

As shown in FIGS. 1 and 2, the bone anchor assembly 10 includes an outer anchor tube 20 and an insert 25. As further shown, the outer anchor tube 20 defines a longitudinal axis 30 of the bone anchor assembly 10, and its tubular shape defines a longitudinal channel 35. In the illustrated embodiment, the outer anchor tube 20 includes a first portion 40 having a first end 42, and a plurality of fingers 45 extending from the first portion 40 generally away from the first end 42. As can be seen in FIGS. 1 and 2, the fingers 45 each have a free end 50 opposite the first portion 40 and terminate in a tip 52 and include edges 54 that are configured to engage bone at the implantation site for securing the bone anchor assembly 10 thereto. Additionally, the first portion 40 includes a plurality of cutouts 55 just proximal to each finger 45 (only one cutout 55 is visible in FIGS. 1 and 2) and a plurality of U-shaped cutouts each forming a tab 57 between the cutouts 55 and the first end 42.

As further shown in FIGS. 1 and 2, each of the fingers 45 is configured to project laterally at an oblique angle relative to the longitudinal axis 30. In addition, the fingers 45 are configured to be deflectable radially inward (i.e., the free ends 50 can be urged toward the longitudinal axis 30) to allow the anchor tube 20 to be disposed within, for example, a tubular cannula during implantation into bone. In the illustrated embodiment, the cutouts 55 operate to provide strain relief in the zone of deflection of the fingers 45. In the illustrated embodiment, four (4) fingers 45 are utilized, although in other embodiments more or fewer than 4 fingers 45 can be present. It will be appreciated that in the various embodiments, the anchor tube 20 includes at least one cutout 55 for each finger 45.

In the illustrated embodiment, the insert 25 includes a head 58 and a shank 60 extending longitudinally from the head 58. As further shown, the insert 25 has a first opening 65 in the head 58, which further includes a radial shoulder 68, a second opening 70 in the shank 60, and a bore 75 extending from the first opening 65 through the second opening 70. In the illustrated embodiment, the first opening 65 is bordered by a chamfered or radiused edge 80, and the second opening 70 is bordered by a chamfered or radiused edge 85.

As shown, the shank 60 is sized so that it can be inserted into the longitudinal channel 35 of the anchor tube 20, with the radial shoulder 68 abutting the first end 42 of the anchor tube 20 first portion 40 when fully inserted. In various embodiments, the fit between the shank 60 and the inner surface of the channel 35 is selected to be sufficiently tight to resist movement of the insert 25 relative to the anchor tube 20. In addition, in the illustrated embodiment, the tabs 57 further operate to engage the insert 25 to further enhance the connection between the insert 25 and the anchor tube 20.

As further shown, the bore 75 is configured to receive the suture element 15, which is configured to be connected to the insert 25. In the illustrated embodiment, the suture element 15 includes a portion 90 disposed within the bore 75 that further includes a pledget 95 having a diameter greater than the diameter of the bore 75 of the insert, such that the suture element 15 cannot readily be pulled proximately through the bore 75 and separated from the bone anchor assembly 10. It is emphasized, however, that the particular technique or structure for connecting the suture element 15 to the insert 25 or the bone anchor assembly 10 is not critical to any of the embodiments of the present invention. For example, in lieu of or in addition to the pledget 95, in various embodiments, a knot, adhesive, or other type of mechanical joining element or technique can be utilized.

The anchor tube 20 and the insert 25 can be made of any number of structurally suitable biocompatible materials. In various embodiments, the anchor tube 20 can be made of a biocompatible alloy or polymeric material. In various embodiments, the anchor tube 20 is made of a superelastic material such as a nickel titanium alloy (e.g., nitinol). Other exemplary materials include titanium, stainless steel, polyetheretherketone, polycarbonate, and combinations thereof. Similarly, the insert 25, in various embodiments, can be made of any number of biocompatible, rigid alloys or polymeric materials, such as titanium, stainless steel, polyetheretherketone, polycarbonate, and combinations thereof. In one embodiment, the anchor tube 20 is made of nitinol, and the insert 25 is made of polyetheretherketone. Various other material combinations can be utilized within the scope of the various embodiments.

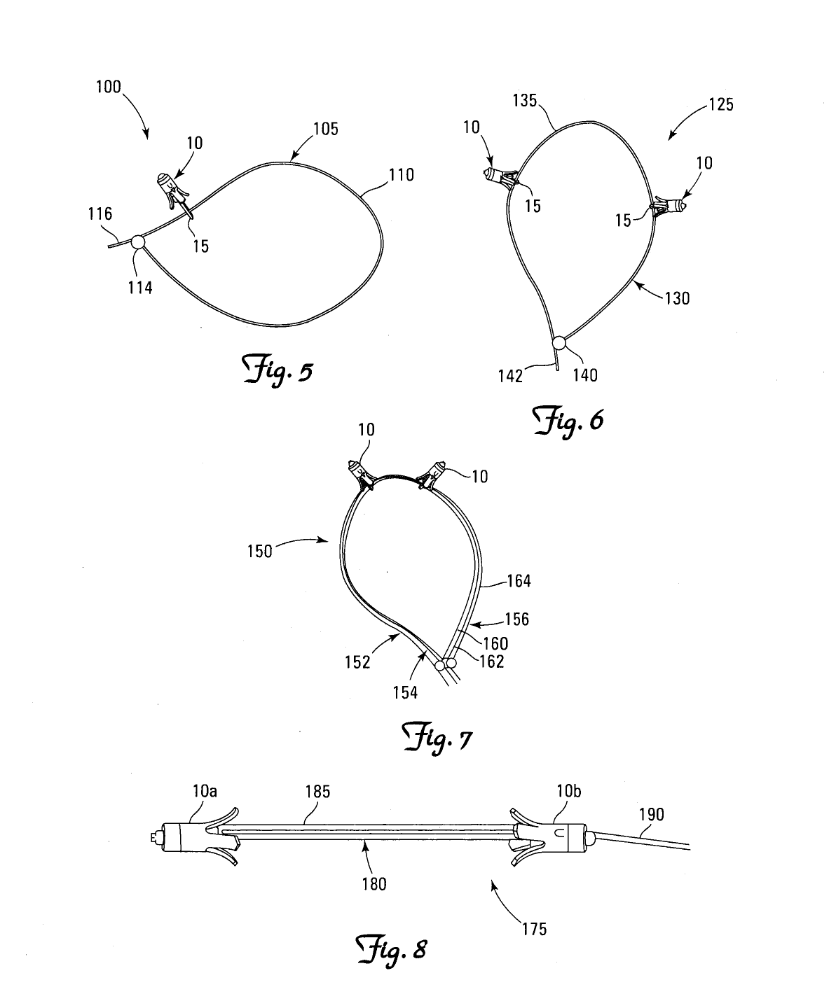

FIGS. 3-4 are elevation views showing implantation of the bone anchor assembly 10 using a cannula 96, according to one embodiment of the present invention. As shown in FIG. 3, in one embodiment, the distal end of the cannula 96, with the bone anchor assembly 10 and suture element 15 disposed therein, is positioned as desired within a bore 98 into the bone 97 at the implantation site. As further shown, when disposed within the cannula 96, the fingers 45 of the anchor tube 20 are deflected radially inward toward the longitudinal axis 30. The bore 98 can be formed by any suitable means, e.g., by use of a bone awl or drill.

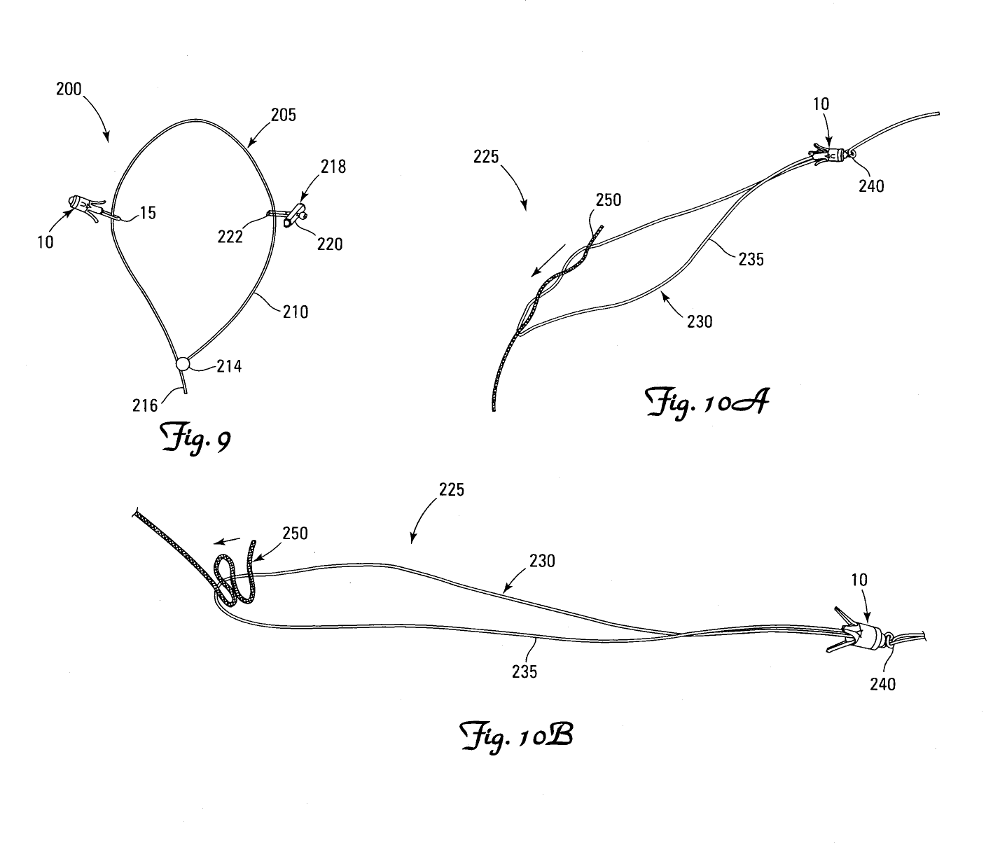

As can be seen in FIG. 4, the cannula 96 can then be withdrawn proximally to release the bone anchor assembly 10 therefrom. For example, a second delivery cannula or push tube 99 can be inserted into the cannula 96 to abut the bone anchor assembly 10 and hold the bone anchor assembly 10 in position while the cannula 96 is withdrawn proximally from the bore 98. As further shown in FIG. 4, once released from the cannula 96, the fingers 45 of the anchor tube 20 self-expand radially outwardly to bear against and engage the bone forming the wall of the bore 98 to secure the bone anchor assembly 10 therein (the engagement of the free end 50, the tip 52 and the edges 54 of the fingers 45 is shown in FIG. 4). Although in FIGS. 3 and 4 a single bone anchor assembly 10 is shown, in various embodiments, as discussed in greater detail below, additional bone anchor assemblies 10 can be disposed serially within the cannula 96 (or other delivery device). In such embodiments, the plurality of bone anchor assemblies 10 can be incorporated into pre-assembled suture assemblies that can take on a variety of configurations for use in various orthopedic procedures. FIGS. 5-10A&B are schematic illustrations of various fixation element constructs according to various exemplary embodiments of the present invention.

FIG. 5 is a schematic illustration of a fixation element 100 according to one embodiment. As shown, the fixation element 100 includes a suture assembly 105, which includes an adjustable suture loop 110, an adjustable knot 114, and a proximal suture length 116 used to tighten the suture loop 110. As further shown, a single bone anchor assembly 10 is slidably coupled to the suture loop 110 by the suture element 15 which is connected to the bone anchor assembly 10 in the manner described above. In the illustrated embodiment, the suture element 15 is in the form of a loop through which the suture material forming the suture loop 110 is passed, thus allowing the bone anchor assembly 10 and suture element 15 to slide along the suture loop 110 as the suture loop is tightened during the particular orthopedic procedure.

FIG. 6 is a schematic illustration of a fixation element 125, which as shown includes a suture assembly 130 having an adjustable suture loop 135, an adjustable knot 140, and a proximal suture length 142. As further shown, a pair of bone anchor assemblies 10 are slidably coupled to the suture loop 135 by respective suture elements 15. Aside from the addition of a second bone anchor assembly 10, the fixation element 125, and its constituent components, operates and is constructed in substantially the same or an identical manner as the fixation element 100. As discussed previously, the fixation element 125 can be pre-loaded into a delivery tool (not shown) with the bone anchor assemblies 10 disposed serially within a delivery cannula, in the manner discussed above with respect to FIGS. 3 and 4.

FIG. 7 is a schematic illustration of an alternative fixation element 150. In the illustrated embodiment, the fixation element 150 includes a pair of bone anchors 10 and corresponding suture elements 15 similar or identical to those discussed previously herein. As further shown, the fixation element 150 includes three suture assemblies 152, 154, 156 each including, respectively, adjustable suture loops 160, 162 and 164, which can each be constructed in substantially the same or a similar manner as the suture loops 110 and 130 discussed above. In the illustrated embodiments, the bone anchor assemblies 10 are slidably coupled to the combined suture loops 160, 162, 164 by their respective suture elements 15. The combination of the three suture assemblies 152, 154, 156 provides a robust, high-strength suture construct.

FIG. 8 is a schematic illustration of another fixation element 175 according to yet another embodiment. As shown in FIG. 8, the fixation element 175 includes a suture assembly 180 and bone anchor assemblies 10a, 10b. In the illustrated embodiment, the suture assembly 180 includes an adjustable suture loop 185 and a proximal suture length 190, which as discussed previously with respect to similar features on other embodiments, can be manipulated to tighten the suture loop 185. The bone anchor assembly 10a is slidably coupled to the suture loop 185, while the anchor assembly 10b is fixedly connected to the suture material making up the suture loop 185 (i.e., cannot slide relative to the suture loop 185). In various embodiments, the bone anchor assemblies 10a, 10b may be disposed, respectively, distally and proximally within a delivery cannula, such that the adjustable bone anchor assembly 10a will be deployed first followed by the fixed bone anchor assembly 10b. In other embodiments, the fixed bone anchor assembly 10b will be disposed distally of the slidable bone anchor assembly 10a in the delivery tool. The specific configuration and orientation of the respective bone anchor assemblies 10a, 10b can be varied depending on the particular orthopedic procedure in which the fixation element 175 is used.

FIG. 9 is a schematic illustration of a fixation element 200 according to one embodiment. As shown, the fixation element 200 includes a suture assembly 205, which includes an adjustable suture loop 210, an adjustable knot 214, and a proximal suture length 216 used to tighten the suture loop 210. As further shown, a single bone anchor assembly 10 is slidably coupled to the suture loop 210 by the suture element 15 which is connected to the bone anchor assembly 10 in the manner described above. As further shown, the fixation element 200 includes a tissue anchor assembly 218 coupled to the suture loop 210. In the illustrated embodiment, the tissue anchor assembly 218 includes a tissue anchor 220 and a suture element 222, which as shown is in the form of a loop through which the suture material forming the suture loop 210 is passed, thus allowing the tissue anchor assembly 218 to slide along the suture loop 210 as it is tightened. In various embodiments, either the bone anchor assembly 10 or the tissue anchor assembly 218 can be fixedly connected to the suture loop 210 in lieu of the slidable coupling arrangement shown. The tissue anchor assembly 218 is configured to be secured to soft tissues (e.g., connective tissue, muscle, or fascia). In various embodiments, the tissue anchor 220 can be formed by a variety of suitable, rigid or semi-rigid polymeric or metallic materials (e.g., polyetheretherketone, PET, titanium, and the like).

FIGS. 10A-10B are schematic illustrations of a fixation element 225 according to yet another embodiment of the present invention. As shown, the fixation element 225 includes a suture assembly 230, which includes an adjustable suture loop 235 and an adjustable knot 240. A bone anchor assembly 10 is slidably coupled to the suture loop 235, and a tissue anchor 250 is coupled to the suture loop 235 opposite the adjustable knot 240. The tissue anchor 250 is constructed of suture material, and is formed by passing the suture material forming the suture loop 235 through the suture material of the tissue anchor 250 at multiple locations along the length of the tissue anchor 250. In use, when the suture loop 235 is tightened with the tissue anchor 250 bearing against the tissue to which it is to be secured, the tissue anchor 250 will tend to bunch up and laterally expand, thereby assuming a deployed configuration in which it will bear against the tissue without passing therethrough. FIG. 10A shows the tissue anchor 250 in its initial, undeployed state, while FIG. 10B shows the tissue anchor 250 in its laterally expanded deployed state.

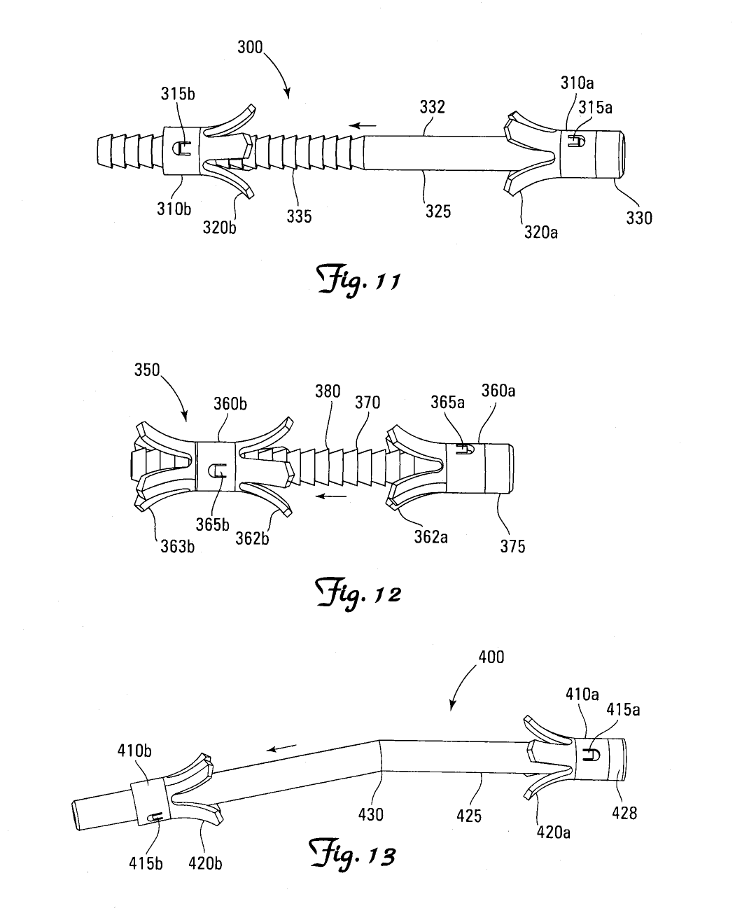

FIG. 11 is an alternative bone anchor assembly 300 according to yet another embodiment of the present invention. As shown in FIG. 11, the bone anchor assembly 300 includes a pair of anchor tubes 310a, 310b oriented in opposite directions from one another. Each of the anchor tubes 310a, 310b can be configured in substantially the same or an identical way to the anchor tube 20 of the bone anchor assembly 10. As such, the anchor tube 310a includes at least one tab 315a and plurality of radially deflectable fingers 320a, corresponding to the tab 57 and the fingers 45 of the anchor tube 20. Similarly, the anchor tube 310b includes at least one tab 315b and a plurality of deflectable fingers 320b, also corresponding to the tab 57 and the fingers 45 of the anchor tube 20. As further shown, the bone anchor assembly 300 includes an elongated insert 325 having a head 330, a shank 332 extending longitudinally from the head 330, and a plurality of serrations 335 on a portion of the shank 332. As further shown, shank 332 is disposed within the tubular anchor tubes 310a, 310b. In the illustrated embodiment, the anchor tube 310a is slidable along the shank 332 with the head 330 of the shank 332 delimiting movement of the anchor tube 310a due to the diameter of the head 330 being greater than the inner diameter of the anchor tube 310a.

As further shown, the anchor tube 310b is disposed along the length of the shank 332 including the serrations 335, and is oriented with its fingers 320b facing the fingers 320a of the anchor tube 310a. As can be seen in FIG. 11, each serration includes a surface extending at an oblique angle with respect to the longitudinal axis of the bone anchor assembly 300, and another surface extending generally orthogonal to the longitudinal axis and oriented generally toward the head 330 of the insert 325. Due to the relative orientations of the anchor tubes 310a, 310b, the shank 332 can be pulled through the anchor tube 310b so as to urge the anchor tubes 310a, 310b toward one another (thus applying tension between two bones or bone regions in which the anchor tubes 310a, 310b are embedded. At the same time, reverse movement of the shank 332 is inhibited by engagement of the tab(s) 315b with the serration surface oriented orthogonally to the longitudinal axis. This arrangement allows a desired amount of tension to be maintained between the bones or bone regions to which the anchor tubes 310a, 310b are secured. In the various embodiments, the anchor tubes 310a, 310b and the shank 332 can be made of substantially the same or identical materials as the anchor tube 20 and the insert 25 of the bone anchor assembly 10.

FIG. 12 is an alternative bone anchor assembly 350 according to yet another embodiment of the present invention. As shown in FIG. 12, the bone anchor assembly 350 includes a pair of anchor tubes 360a, 360b oriented in opposite directions from one another and each including, respectively, at least one tab 365a, 365b, and a plurality of deflectable fingers 362a, 362b disposed such that the fingers 362a are oriented toward the fingers 362b. As further shown, in the illustrated embodiment, the anchor tube 360b further includes a plurality of deflectable fingers 363b positioned opposite the fingers 362b, and thus oriented in the same general direction as the fingers 362a of the anchor tube 360a. The bone anchor assembly 350 further includes an insert 370 having a head 375 and a shank including a plurality of serrations 380. The insert 370 extends through the anchor tubes 360a, 360b, and the head 375 and the serrations operate in the same manner as the corresponding features of the bone anchor assembly 300 discussed previously.

FIG. 13 is a schematic illustration of a bone anchor assembly 400 according to another embodiment of the present invention. As shown, the bone anchor assembly 400 includes a pair of anchor tubes 410a, 410b oriented in opposite directions from one another. Each of the anchor tubes 410a, 410b can be configured in substantially the same or an identical way to the anchor tube 20 of the bone anchor assembly 10. As such, the anchor tube 410a includes at least one tab 415a and plurality of radially deflectable fingers 420a, corresponding to the tab 57 and the fingers 45 of the anchor tube 20. Similarly, the anchor tube 410b includes at least one tab 415b and a plurality of deflectable fingers 420b, also corresponding to the tab 57 and the fingers 45 of the anchor tube 20. As further shown, the bone anchor assembly 400 includes an elongated insert 425 having a head 428, and a shank extending longitudinally from the head 428. The particular shank shown includes a bend 430 at a predetermined location along its length. The bend 430 in the shank provides enhanced flexibility in orienting the bone anchor assembly 400 to provide the desired effect. In various embodiments, the insert 425 can also include serrations (not shown) along its length, similar or identical to the inserts of the bone anchor assemblies previously described. In addition, in some embodiments, the anchor tube 410b can be configured in substantially the same or an identical manner as the anchor tube 360b discussed above (e.g., with two arrangements of deflectable fingers extending in opposite directions from one another).

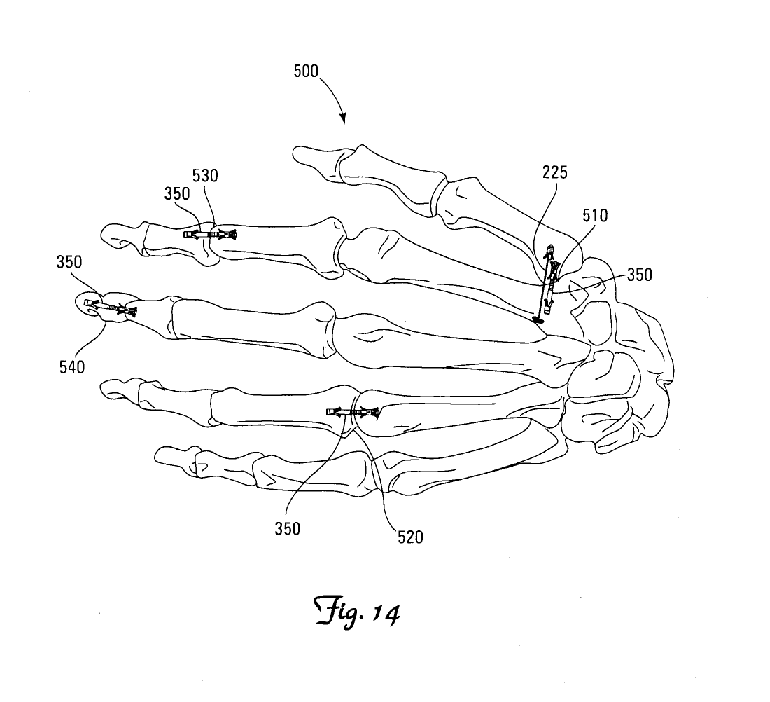

FIG. 14 is a schematic illustration of a model of the skeletal system of a human hand 500 showing exemplary therapeutic applications of various embodiments of the present invention. As shown in FIG. 14 at 510, in one exemplary embodiment, a bone anchor assembly 350 can be deployed in combination with a bone anchor/suture assembly—in this case the fixation element 225, in a procedure to repair the carpal metacarpal (CMC) joint. In such an embodiment, a plurality of bone bores can be formed into or through the bones and the bone anchor assembly 360 and the fixation element 225 can be deployed through such bone bores using a suitable delivery tools and techniques, and thereafter tightened to complete the desired orthopedic procedure.

In another example, as further shown in FIG. 14, one of the bone anchor assemblies 350 can also be utilized to accomplish or facilitate fusion of the metacarpophalangeal (MCP) joint 520, the proximal interphalangeal (PIP) joint 530 and/or the distal interphalangeal (DIP) joint 540. In these embodiments, a bone bore can be formed across the respective joints 520, 530, 540 and the bone anchor assembly 350 deployed across the joint through this bore as shown in FIG. 14. In various embodiments, once the bone anchor assembly 350 is inserted into and across the respective joint 520, 530, 540 and the corresponding anchor tubes embedded in the respective bone masses, the insert of the bone anchor assembly 350 can be pulled proximally so as to urge the anchor tubes toward one another thereby accomplishing or aiding in fixation of the joint 520, 530 or 540. It will be appreciated that, in other embodiments, one or more of the additional bone anchor assemblies and/or fixation elements described herein can also be advantageously in the same or similar orthopedic procedures.

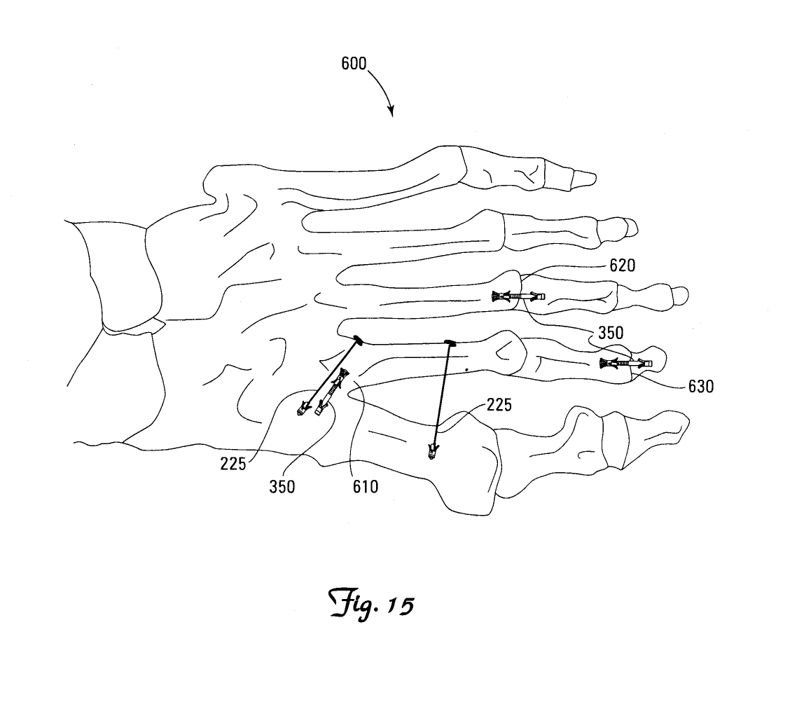

FIG. 15 is a schematic illustration of a model of the skeletal system of a human foot 600 illustrating additional exemplary therapeutic applications of various embodiments of the present invention. As shown in FIG. 15 at 610, one or more bone anchor assemblies 350 can be deployed in the bones of the foot 600 in combination with one or more additional anchor/suture assemblies, in this case, the fixation element 225, in hallux valgus and/or hammertoe repair procedures. As further shown in FIG. 15, the bone anchor assembly 350 can also be utilized to accomplish or facilitate fusion of the proximal interphalangeal (PIP) joint 620 and/or the distal interphalangeal (DIP) joint 630 of the foot 600. In these embodiments, a bone bore can be formed across the respective joints 620, 630 and the bone anchor assembly 350 deployed across the joint through this bore. In various embodiments, once the bone anchor assembly 350 is inserted into and across the respective joint 620, 630 and the corresponding anchor tubes embedded in the respective bone masses, the insert of the bone anchor assembly 350 can be pulled proximally so as to urge the anchor tubes toward one another thereby accomplishing or aiding in fixation of the joint 620 or 630, as the case may be. It will be appreciated that, in other embodiments, one or more of the additional bone anchor assemblies and/or fixation elements described herein can also be advantageously in the same or similar orthopedic procedures.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. An implantable bone anchor assembly comprising:
an outer anchor tube defining a longitudinal axis of the bone anchor assembly and a longitudinal channel, the outer anchor tube having a first portion including a first end, and a plurality of fingers circumferentially spaced from one another about the longitudinal axis, each of the fingers extending from the first portion and having a free end, wherein the fingers are configured to project laterally at an oblique angle relative to the longitudinal axis, and are further configured to be deflectable radially inward to allow the outer anchor tube to be disposed within a tubular cannula for deployment; and
a tubular insert having a head including an opening therein, a shank extending from the head having an open end, and a longitudinal bore extending from the opening in the head to the open end of the shank, the shank sized to be received in the longitudinal channel,
wherein the outer anchor tube and the tubular insert are configured to be secured together upon complete insertion of the shank into the longitudinal channel.

2. The bone anchor assembly of claim 1, wherein the opening in the head and the open end of the shank are defined by radiused or chamfered edges.

3. The bone anchor assembly of claim 1, wherein the head of the insert has a radial shoulder that abuts the first end of the outer anchor tube when the shank is fully inserted into the longitudinal channel of the outer anchor tube.

4. The bone anchor assembly of claim 1, wherein the anchor tube includes a plurality of tabs configured to engage the insert to couple the insert and the outer anchor tube together when the shank is fully inserted into the longitudinal channel.

5. The bone anchor assembly of claim 1, wherein the outer anchor tube is made from a shape memory alloy.

6. The bone anchor assembly of claim 1, wherein the fingers of the outer anchor tube are configured to engage bone when implanted.

7. The bone anchor assembly of claim 1, further comprising an adjustable suture assembly including a first portion extending within the bore of the insert and configured to engage the insert to resist withdrawal of the first portion from the bore of the insert after implantation.

8. The bone anchor assembly of claim 1 wherein the outer anchor tube is a first outer anchor tube, and further comprising a second outer anchor tube having a longitudinal channel, and a plurality of fingers circumferentially spaced from one another about the longitudinal axis, each of the fingers of the second outer anchor tube having a free end and being configured to project laterally at an oblique angle relative to the longitudinal axis of the second outer anchor tube and to be deflectable radially inward to allow the second anchor tube to be disposed within a tubular cannula for deployment, wherein the shank of the insert is sized to be received within the longitudinal channel of the second outer anchor tube.

9. The bone anchor assembly of claim 8, wherein the shank includes a plurality of serrations along a portion of its length, each serration including a first surface extending at an oblique angle with respect to the longitudinal axis of the bone anchor assembly and a second surface extending generally orthogonal to the longitudinal axis and oriented generally toward the head of the insert, wherein the second outer anchor tube is disposed along a portion of the shank including the serrations, and wherein the serrations and the second outer anchor tube cooperate to permit relative translation of the insert and the second outer anchor tube in a first direction, and to inhibit relative translation of the insert and the second outer anchor tube in a second direction opposite the first direction.

10. A fixation element for an orthopedic procedure, the fixation element comprising:
a bone anchor assembly including:
an outer anchor tube defining a longitudinal axis of the bone anchor assembly and a longitudinal channel, the outer anchor tube having a first portion including a first end, and a plurality of fingers circumferentially spaced from one another about the longitudinal axis, each of the fingers extending from the first portion and having a free end, wherein the fingers are configured to project laterally at an oblique angle relative to the longitudinal axis, and are further configured to be deflectable radially inward to allow the outer anchor tube to be disposed within a tubular cannula for deployment; and
a tubular insert having a head including an opening therein, a shank extending from the head having an open end, and a longitudinal bore extending from the opening in the head to the open end of the shank, the shank sized to be received in the longitudinal channel,
wherein the outer anchor tube and the tubular insert are configured to be secured together upon complete insertion of the shank into the longitudinal channel;
a suture assembly coupled to the bone anchor assembly and including:
an adjustable suture loop; and
a knot configured for facilitating tightening of the suture loop,
wherein the bone anchor assembly is coupled to the adjustable suture loop.

11. The fixation element of claim 10, wherein the bone anchor assembly is slidably coupled to the suture loop.

12. The fixation element of claim 11, further comprising a suture element connected to the bone anchor assembly and extending at least partially within the longitudinal bore of the insert, wherein the suture element is formed as a loop and is configured to slidably couple the bone anchor assembly to the adjustable suture loop.

13. The fixation element of claim 10 further comprising a second bone anchor assembly including:
a second outer anchor tube defining a longitudinal axis of the second bone anchor assembly and a longitudinal channel, the second outer anchor tube having a first portion including a first end, and a plurality of fingers circumferentially spaced from one another about the longitudinal axis, each of the fingers extending from the first portion and having a free end, wherein the fingers are configured to project laterally at an oblique angle relative to the longitudinal axis, and are further configured to be deflectable radially inward to allow the second outer anchor tube to be disposed within a tubular cannula for deployment; and
a second tubular insert having a head including an opening therein, a shank extending from the head having an open end, and a longitudinal bore extending from the opening in the head to the open end of the shank, the shank sized to be received in the longitudinal channel,
wherein the second outer anchor tube and the second tubular insert are configured to be secured together upon complete insertion of the shank of the second tubular insert into the longitudinal channel of the second outer anchor tube, and
wherein the second bone anchor assembly is coupled to the adjustable suture loop.

14. The fixation element of claim 13, wherein the bone anchor assembly and the second bone anchor assembly are slidably coupled to the adjustable suture loop.

15. The fixation element of claim 13, wherein one of the bone anchor assembly and the second bone anchor assembly is slidably coupled to the adjustable suture loop, and wherein the other of the bone anchor assembly and the second bone anchor assembly is fixedly coupled to the adjustable suture loop.

16. The fixation element of claim 10, further comprising a tissue anchor coupled to the adjustable suture loop.

17. The fixation element of claim 16, wherein the tissue anchor is formed from a length of suture material, and wherein the adjustable suture loop passes through the length of suture material of the tissue anchor at a plurality of locations therein.

18. A method of deploying a fixation element for use in an orthopedic repair procedure, the method comprising:
forming a first bore within a first bone proximate tissue to be repaired;
positioning a cannula within the bore, the cannula releasably receiving at least a portion of a fixation element including a bone anchor assembly including:
an outer anchor tube defining a longitudinal axis of the bone anchor assembly and a longitudinal channel, the outer anchor tube having a first portion including a first end, and a plurality of fingers circumferentially spaced from one another about the longitudinal axis, each of the fingers extending from the first portion and having a free end, wherein the fingers are configured to project laterally at an oblique angle relative to the longitudinal axis, and are further configured to be deflectable radially inward, wherein the cannula retains the fingers in the inward deflected configuration for delivery; and
a tubular insert having a head including an opening therein, a shank extending from the head having an open end, and a longitudinal bore extending from the opening in the head to the open end of the shank, the longitudinal bore configured to receive a portion of a suture assembly for an orthopedic treatment, the shank sized to be received in the longitudinal channel, wherein the outer anchor tube and the tubular insert are configured to be locked together upon complete insertion of the shank into the longitudinal channel; and
removing the cannula while leaving the bone anchor assembly within the bore, wherein removing the cannula allows the fingers to project radially outward at an oblique angle to engage a surface within the bore.

19. The method of claim 18, wherein the fixation element further includes an adjustable suture assembly including a first portion extending at least partially within the longitudinal bore of the insert coupled to the insert, and a second portion extending from or coupled to the first portion and including an adjustable suture loop.

20. The method of claim 18, wherein the fixation element further includes a second bone anchor assembly, and wherein the bone anchor assembly and the second bone anchor assembly are pre-loaded into the cannula prior to deployment of the bone anchor assembly and the second bone anchor assembly.

21. The method of claim 20, further comprising:
forming a second bore within the first bone or a second bone proximate tissue to be repaired;
positioning the cannula within the second bore; and
removing the cannula while leaving the second bone anchor assembly in the second bore.
